(19) United States
(12) Patent Application Publication
(10) Pub. No.: US 2006/0079786 A1
(43) Pub. Date: Apr. 13, 2006

(54) Blood flow meter and flow probe therefor

(75) Inventors: Alyse Renee Stofer, Woodbury, MN (US); Tanya Shipkowitz, St. Paul, MN (US); John M. Yates, St. Paul, MN (US); Justin Vanhee, Minneapolis, MN (US); Robert V. Brockway, Maple Grove, MN (US); Robert Busch, Taylors Falls, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Transoma Medical, Inc., Saint Paul, MN (US)

(21) Appl. No.: 11/249,069

(22) Filed: Oct. 11, 2005

Related U.S. Application Data
(60) Provisional application No. 60/617,306, filed on Oct. 8, 2004.

(51) Int. Cl. A61B 8/14

(52) U.S. Cl. 600/465

(57) ABSTRACT

Flow probe designs that are relatively atraumatic to blood vessels and therefore are suitable for chronic implantation.

### FIELD OF THE INVENTION

The present invention generally relates to flow meters for measuring volumetric fluid flow through a conduit, such as implantable flow meters for measuring blood flow in a blood vessel.

### BACKGROUND OF THE INVENTION

There are many applications in clinical and research medicine in which measurement or estimation of volumetric blood flow within a blood vessel is desirable. Examples of methods that may be used to make such measurements include transit time, pulse Doppler, and continuous wave Doppler measurements. Such methods may, for example, be used to measure blood flow velocity and thereby estimate volumetric blood flow.

An exemplary method of transit time measurement of blood flow velocity is discussed in U.S. Pat. No. 5,865,749 the entire disclosure of which is hereby incorporated by reference. In that exemplary method, first and second transducers are configured for ultrasonic communication therebetween via an acoustic reflector. A first ultrasonic impulse is launched from the first transducer, reflected from the reflector, and received at the second transducer. A second ultrasonic impulse is launched from the second transducer, reflected from the reflector, and received at the first transducer. In this exemplary method, the first impulse has a directional component in the same direction as the blood flow in a blood vessel, and the second impulse has a directional component opposite the direction of blood flow in the blood vessel. As a result, a travel time of the second impulse from the second transducer to the first transducer is longer than a travel time of the first impulse from the first transducer to the second transducer. Blood flow velocity may be calculated from the difference in transit times of the first and second impulses.

Blood flow velocity may also be measured using the Doppler effect. In some exemplary methods, single frequency ultrasonic energy is transmitted into an area of tissue containing the blood flow to be measured. This insonification of the area is typically referred to as illumination. Resulting ultrasonic energy is reflected, or backscattered, from the illuminated area. Energy reflected from moving targets, such as fluid and blood cells, will be shifted in frequency from the illuminating frequency according to the well-known Doppler effect. The Doppler shifted frequency provides a measure of the blood flow velocity.

In clinical and research applications, it is often necessary to study blood flow for an extended period of time. Thus, in ambulatory living organisms, such as animal or human subjects, there is a need in the art to provide a battery-powered blood flow meter for measuring blood flow velocity for an extended period of time, allowing a human or animal patient freedom of movement during the study and minimizing the need for supervision by the clinician. There is also a need in the art to provide a small, low power blood flow meter that is suitable for implantation in a human or animal subject. There is a further need in the art to provide a blood flow meter with an associated flow probe that is atraumatic to the blood vessel it surrounds.

### SUMMARY OF THE INVENTION

To address this need and provide additional advantages, the present invention provides, by way of example, not limitation, flow probe designs that are relatively atraumatic to blood vessels and therefore are suitable for chronic implantation.

### BRIEF DESCRIPTION OF THE DRAWINGS

FIG. 1 schematically illustrates an example of a blood flow meter system according to a first embodiment;

FIGS. 2A-2D illustrate a second embodiment of a housing design for a flow probe;

FIGS. 3A-3E illustrate a third embodiment of a housing design for a flow probe;

FIGS. 4A-4E illustrate a forth embodiment of a housing design for a flow probe;

FIGS. 5A-5E illustrate a fifth embodiment of a housing design for a flow probe;

FIGS. 6A-6E illustrate a sixth embodiment of a housing design for a flow probe;

FIGS. 7A-7D illustrate an alternative embodiment of a coupling member for a flow probe;

FIG. 8 schematically illustrates another embodiment of a flow probe; and

FIG. 9 schematically illustrates yet another embodiment of a flow probe.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

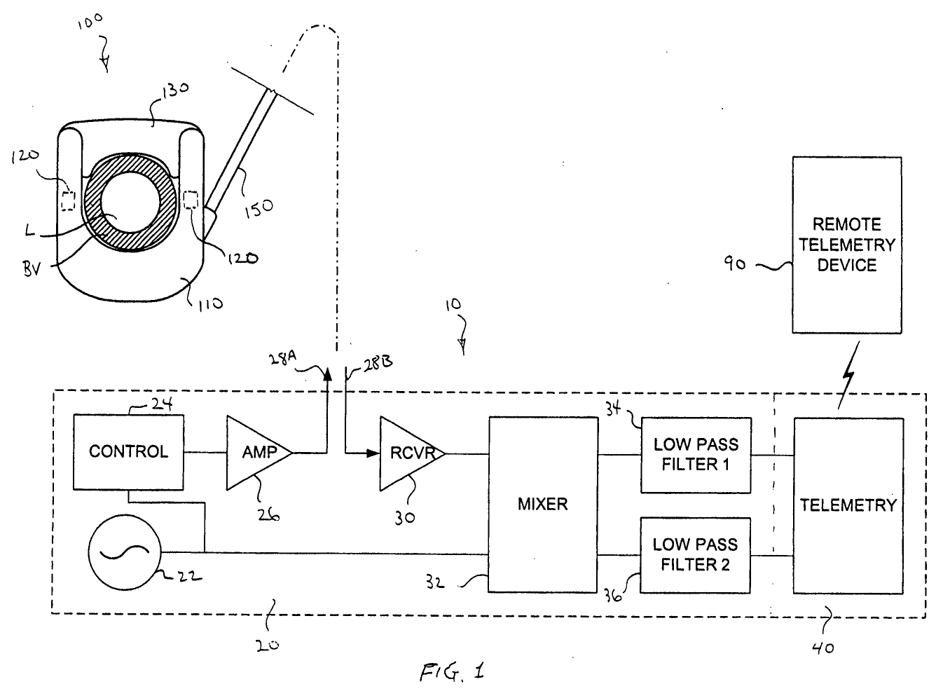

With reference to FIG. 1, an exemplary embodiment of a flow meter system is shown schematically. The flow meter system may be used to measure fluid flow in a conduit such as blood flow in a blood vessel. For purposes of illustration, not necessarily limitation, the flow meter system is described in the context of an implantable blood flow meter.

The blood flow meter system may include an implantable electronics module 10, which may generally include a meter circuit 20 and a telemetry circuit 40. The components of the electronics module 10 may be unified or partitioned as desired. The electronics module 10 may be disposed in a biocompatible housing to facilitate acute or chronic implantation, and may be hermetically sealed to facilitate chronic implantation.

The telemetry circuit 40 may wirelessly communicate with a remote telemetry device 90, which may comprise an external receiver, transceiver, or repeater, for example. The remote telemetry device 90 may communicate with other systems and devices such as a network, a computer, etc. Alternatively, the remote telemetry device 90 may comprise another implanted device such as an implantable pacemaker, defibrillator, flow pump (e.g., ventricular assist device), infusion pump, or other therapeutic device.

The blood flow meter system may further include an implantable flow probe 100, which may be connected to the electronics module 10 by cable 150. The flow probe 100 generally includes a housing portion 110 containing one or more flow sensors 120. The housing 110 is sized and shaped to at least partially surround a conduit such as a blood vessel BV to measure the flow of a fluid such as blood flowing in the lumen L thereof. The housing 110 may define an open geometry (e.g., V-shaped, U-shaped, and other similar shapes) to receive the blood vessel through the opening during implantation. A coupling member 130 may be connected to the housing 110 and may be sized and shaped to at least partially surround the conduit and to close the open portion of the housing 110, thus securing the probe 100 about the blood vessel BV.

The housing 110 and the coupling member 130 may comprise molded polymeric material. The transducers 120 may be insert molded in the housing 110. The material of the housing 110 may be relatively rigid to maintain alignment of the transducers 120, and may be made more rigid with embedded reinforcement structure such as fiber glass. Such molding process may be performed by Transonic Systems, Inc., Ithaca, N.Y.

The flow sensors 120 may comprise ultrasound transducers, for example, such as piezoelectric crystals that convert electrical energy to high-power ultrasonic energy. In one embodiment, two transducers in a pair are positioned opposing one another so that they have a common field of view, and the transducers contemporaneously transmit signals through the blood vessel to be received by the opposite crystal. In another embodiment, two pairs of transducers are used with one transducer in each pair positioned opposing one another so that they have a common field of view, and the transducers transmit signals through the blood vessel to be received by an opposing crystal.

For example, a transducer pair may be positioned diametrically opposite and longitudinally spaced from each other. Generally, as used herein, longitudinal refers to a direction parallel to the length of the lumen L, and lateral refers to a direction parallel to the radius of the lumen L. So, for example, the blood vessel BV in FIG. 1 is shown in lateral cross section.

The transducers may be located and/or aligned in any number of ways in order to achieve desired measurements such as, for example, transit time flow measurement, pulse Doppler, and continuous wave Doppler measurements. It is understood that other measurements are possible without departing from the scope of the present invention. It is also understood that the sensor housing is not limited to housing transducers. Furthermore, the number, type and operation of devices which are used may vary without departing from the scope of the present invention.

Other aspects (e.g., material selection, dimensions, etc.) of the flow probe 100 may be found in U.S. Pat. No. 6,709,430 to Doten et al., which discloses an alternative flow probe design, the entirety of which is incorporated herein by reference.

With continued reference to FIG. 1, the meter circuit 20 may comprise an implantable meter as described in U.S. Pat. No. 6,626,838 to Doten et al., the entire disclosure of which is incorporated by reference. Alternatively, the meter circuit 20 may comprise a variety of commercially available circuit designs, such as those from Transonic Systems, Inc., Ithaca, N.Y. However, such commercially available systems are typically not implantable, and therefore may be worn by the patient or animal test subject with the cable 150 extending transcutaneously. Preferably, however, the electronics module 10 is implantable (i.e., small, battery powered, and enclosed in a hermetically sealed biocompatible housing, for example) and includes a telemetry circuit 40.

In the example shown in FIG. 1, the meter circuit 20 comprises a strobed circuit to conserve battery power and increase longevity, which makes it particularly suitable for chronic implant applications. The meter circuit 20 includes an oscillator 22, such as a sine or square wave oscillator operating at a carrier frequency in an ultrasonic region of the frequency spectra, typically in the 5-20 MHz range, though other frequencies are also possible. The ultrasonic sine or square wave output signal of oscillator 22 may be referred to as a carrier signal, and is electrically coupled to a control circuit 24. Control circuit 24 produces at control circuit output a resulting electrical strobed ultrasonic-frequency signal, which is electrically coupled to power amplifier 26. Amplifier 26 produces a resulting electrical strobed amplified ultrasonic-frequency signal which is electrically coupled through node 28A to transducer 120 via cable 150. In response, transducer 120 provides ultrasonic energy that is mechanically or acoustically coupled to blood and tissue including blood vessel BV and blood flowing in lumen L. As used herein, providing ultrasonic energy, insonifyng, and insonating, are all referred to generally as illuminating.

Illumination of the blood vessel BV results in a reflected Doppler-shifted ultrasound signal, also referred to as a backscattered signal, that is received at the other transducer 120 and converted into a Doppler-shifted electrical signal. The Doppler shifted electrical signal is electrically coupled through node 28B via cable 150 to receiver 30, which provides a buffered Doppler-shifted signal in response thereto.

A mixer 32 receives the buffered Doppler-shifted signal from the receiver 30. The mixer 32 also receives the carrier signal of oscillator 22. The mixer 32 performs a demodulation function by quadrature mixing, producing an in-phase signal (I) and a phase-shifted signal (Q), which is 90 degrees out of phase with respect to the I signal. The I and Q signals each have components that include difference and sum frequency components that are approximately equal to the respective difference and sum of the frequencies of the carrier signal and the buffered Doppler-shifted signal. The I and Q signals may also contain a carrier frequency component, also referred to as carrier feedthrough.

The I signal is electrically coupled to a first low pass filter 34 which removes the carrier feedthrough and the sum frequency components of the I signal, and provides the difference frequency component, which is referred to as the basebanded in-phase Doppler signal or the basebanded I Doppler signal. Similarly, the Q signal is electrically coupled to a second low pass filter 36 which removes the carer feedthrough and the sum frequency components of the Q signal and provides the difference frequency component, which is referred to as the basebanded phase-shifted Doppler signal, or the basebanded Q Doppler signal.

The basebanded I and Q Doppler signals are electrically coupled to telemetry circuit 40. In one embodiment, the basebanded I and Q Doppler signals are re-modulated with a telemetry carer frequency for transmission to the remote telemetry device 90. In another embodiment, an analog velocity output signal is produced, which is encoded, such as by pulse position modulation, for transmission to remote telemetry device 90. Thus, telemetry circuit 40 allows transmission of the signals corresponding to the basebanded I and Q Doppler signals from implanted blood flow meter to a remote telemetry device 90 for further processing. In one embodiment, this further processing includes velocity determination according to the well-known Doppler equation.

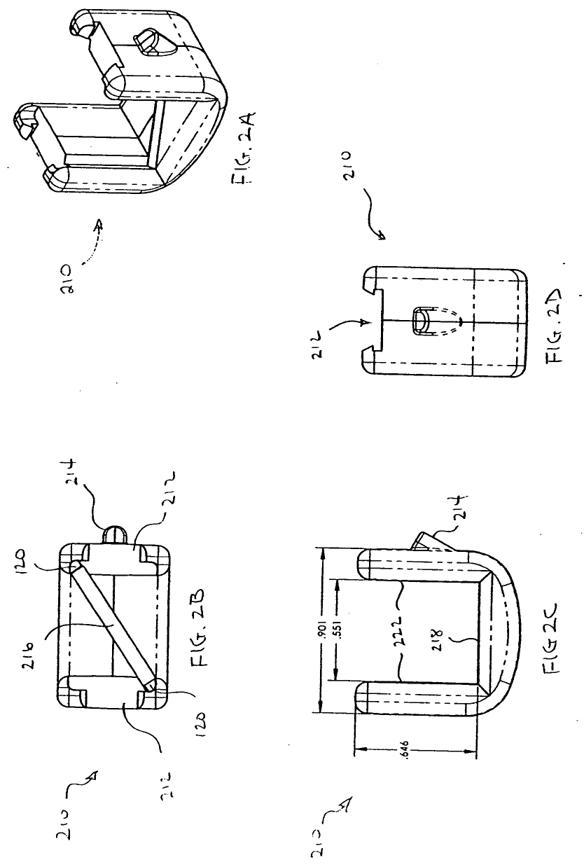

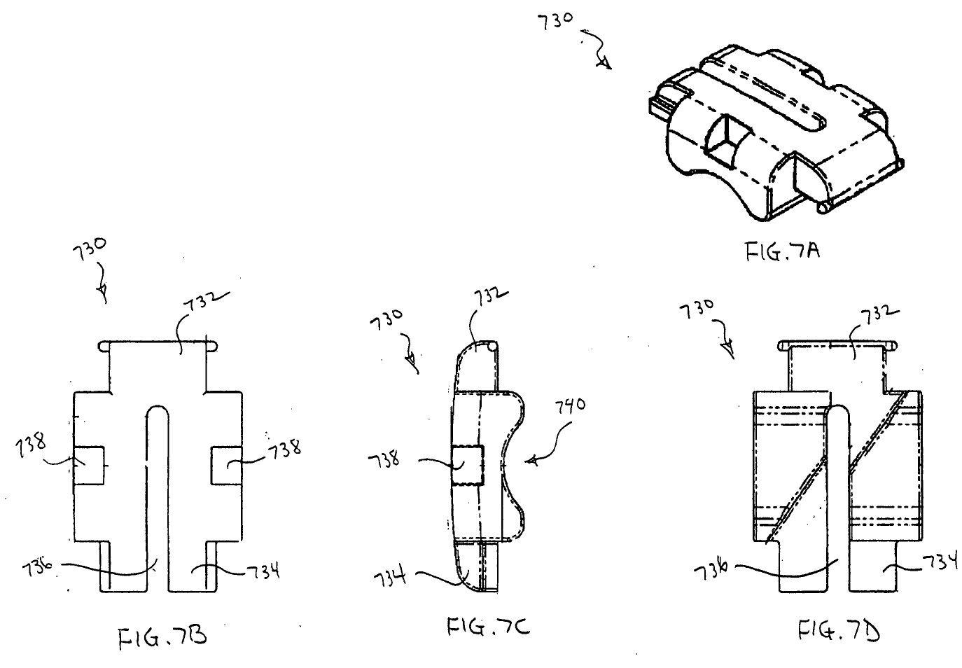

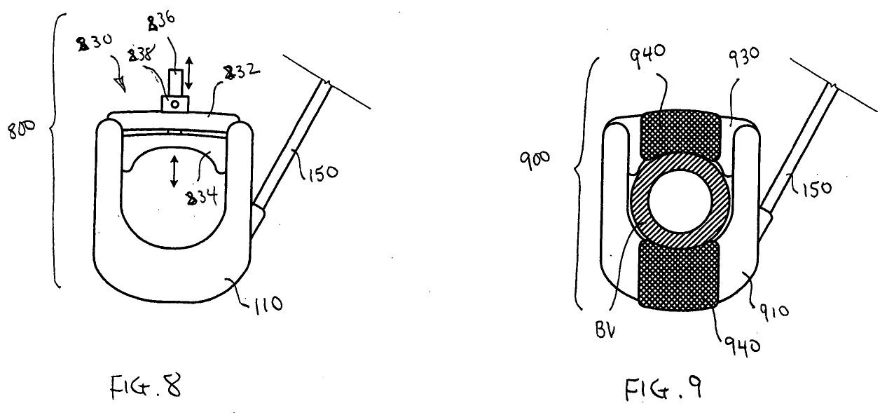

With reference to FIGS. 2A-2D a second embodiment of a housing design 210 for the flow probe 100 is shown schematically. FIG. 2A provides a perspective view, FIG. 2B provides a top view, FIG. 2C provides a front view, and FIG. 2D provides a side view. With specific reference to FIG. 2B, the housing 210 includes two receptacles 212 to receive a coupling member, such as coupling member 730 shown in FIG. 7. Housing 210 also includes a strain relief 214 through which the cable 150 may extend.

A transducer channel 216 extends along the inside surface of the housing 210, including bottom surface 218 and side surfaces 222. Channel 216 functions as a window for the ultrasonic transducers 120 through which the transducers 120 illuminate the blood vessel therein. Channel 216 is shaped to position the transducers 120 diametrically opposite, longitudinally displaced from each other, and facing each other squarely.

The inside surface of the housing 210, including bottom surface 218 and side surfaces 222, may define a square shape as seen in lateral cross section or by frontal view as shown in FIG. 2C. Relative to the circular cross sectional shape of blood vessels, the square shape of the housing 210 interior reduces the amount of contact area therebetween. In some applications, the likelihood of vessel erosion may be reduced.

In each of the housing embodiments described herein, it is anticipated that any gaps between the blood vessel and the interior surface of the housing, including the transducer channel, will fill with biological material post implantation. Alternatively, the gaps and/or channel may be pre-filled with a material such as a gel, a biocompatible foam, or a bone wax, for example. In some useful embodiments, the pre-filled material may acoustically match the blood vessel to avoid interference.

Also in each of the housing embodiments described herein, the receptacles that receive the coupling member may be covered by an atraumatic temporary cap (e.g., silicone cap) or pre-filled with a material (e.g., biodegradable or dissolvable) such as a biodegradable foam, a bone wax, or Manitol, for example. Covering or pre-filling the receptacles (i.e., prior to insertion around a blood vessel) reduces the likelihood of snagging on or otherwise damaging the blood vessel or surrounding tissue. Once the housing is around the blood vessel, the cap or material may be removed to make way for the coupling member.

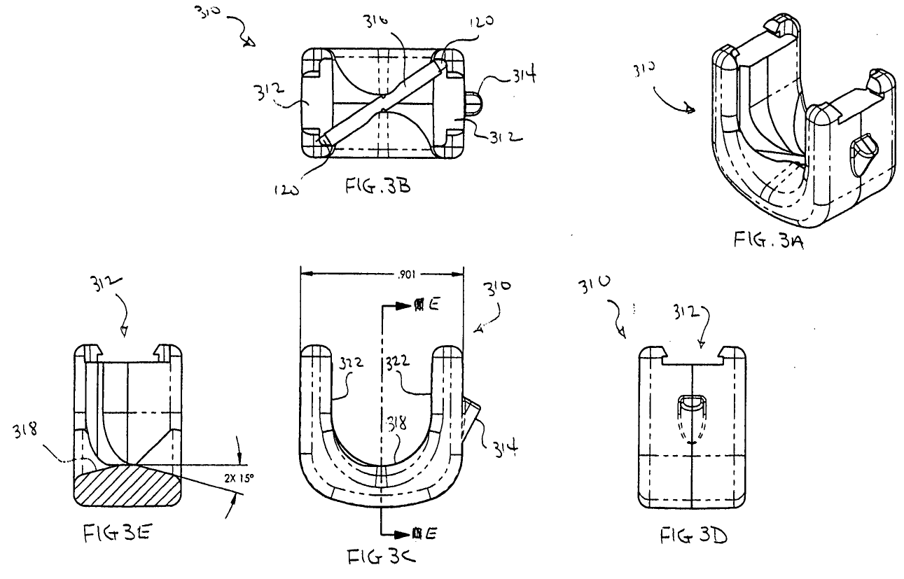

With reference to FIGS. 3A-3E, a third embodiment of a housing design 310 for the flow probe 100 is shown schematically. FIG. 3A provides a perspective view, FIG. 3B provides a top view, FIG. 3C provides a front view, FIG. 3D provides a side view, and FIG. 3E provides a longitudinal cross sectional view taken along line E-E in FIG. 3C. With specific reference to FIG. 3B, the housing 310 includes two receptacles 312 to receive a coupling member, such as coupling member 730 shown in FIG. 7. Housing 310 also includes a strain relief 314 through which the cable 150 may extend.

A transducer channel 316 extends along the inside surface of the housing 310, including bottom surface 318 and side surfaces 322. Channel 316 functions as a window for the ultrasonic transducers 120 through which the transducers 120 illuminate the blood vessel therein. Channel 316 is shaped to position the transducers 120 diametrcally opposite, longitudinally displaced from each other, and facing each other squarely.

The inside surface of the housing 310, including bottom surface 318 and side surfaces 322, may define a V-shape as seen in lateral cross section or by frontal view as shown in FIG. 3C. The interior surface of the housing 310 may have a concave geometry in lateral cross section or frontal view as seen in FIG. 3C, and a convex cured or tapered geometry in longitudinal cross section as seen in FIG. 3E, thereby reducing the likelihood of vessel erosion.

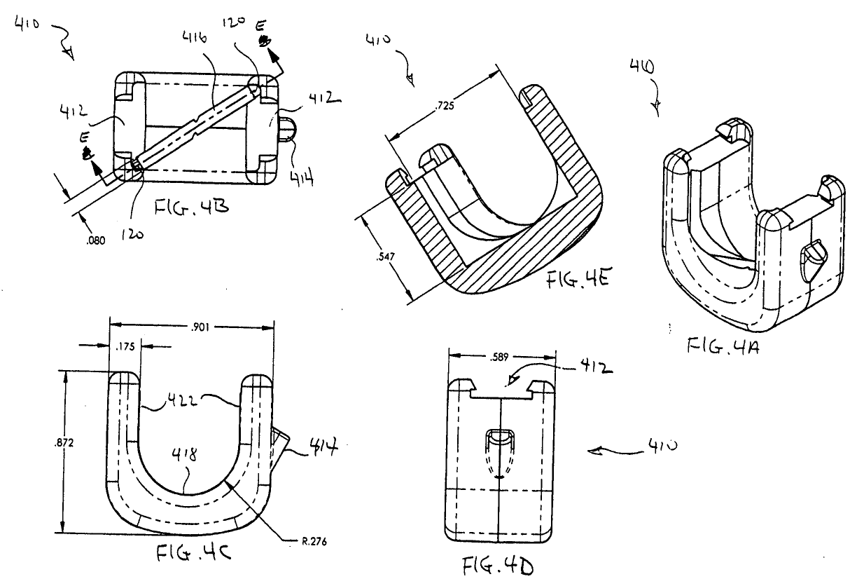

With reference to FIGS. 4A-4E, a forth embodiment of a housing design 410 for the flow probe 100 is shown schematically. FIG. 4A provides a perspective view, FIG. 4B provides a top view, FIG. 4C provides a front view, FIG. 4D provides a side view, and FIG. 4E provides a longitudinal cross sectional view taken along line E-E in FIG. 4B. With specific reference to FIG. 4B, the housing 410 includes two receptacles 412 to receive a coupling member, such as coupling member 30 shown in FIG. 7. Housing 410 also includes a strain relief 414 through which the cable 150 may extend.

A transducer channel 416 extends along the inside surface of the housing 410, including bottom surface 418 and side surfaces 422. Channel 416 functions as a window for the ultrasonic transducers 120 through which the transducers 120 illuminate the blood vessel therein. Channel 416 is shaped to position the transducers 120 diametrically opposite, longitudinally displaced from each other, and facing each other squarely.

The inside surface of the housing 410, including bottom surface 418 and side surfaces 422, may define a V-shape as seen in lateral cross section or by frontal view as shown in FIG. 4C. In other words, the interior surface of the housing 410 may have a concave geometry in lateral cross section or frontal view as seen in FIG. 4C, thereby reducing the likelihood of vessel erosion. As compared to housing 310, housing 410 does not include a longitudinal taper as seen in FIG. 3E.

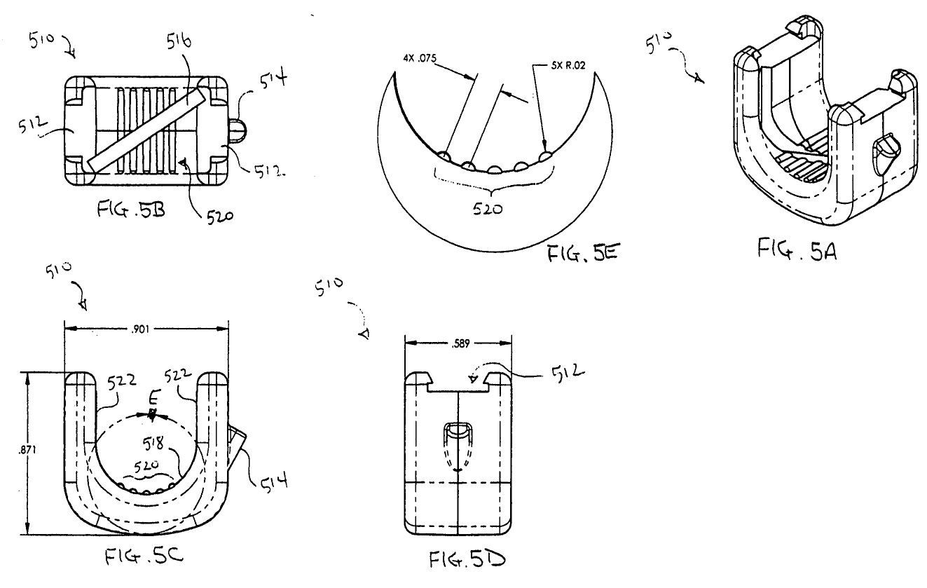

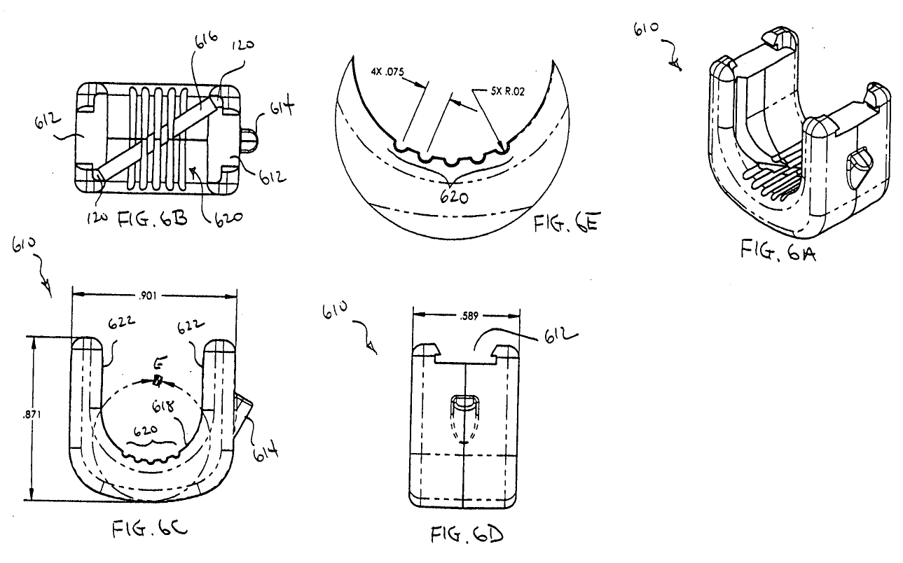

With reference to FIGS. 5A-5E, a fifth embodiment of a housing design 510 for the flow probe 100 is shown schematically. FIG. 5A provides a perspective view, FIG. 5B provides a top view, FIG. 5C provides a front view, FIG. 5D provides a side view, and FIG. 5E provides a detailed view taken along line E in FIG. 5C. With specific reference to FIG. 5B, the housing 510 includes two receptacles 512 to receive a coupling member, such as coupling member 730 shown in FIG. 7. Housing 510 also includes a strain relief 514 through which the cable 150 may extend.

A transducer channel 516 extends along the inside surface of the housing 510, including bottom surface 518 and side surfaces 522. Channel 516 functions as a window for the ultrasonic transducers 120 through which the transducers 120 illuminate the blood vessel therein. Channel 516 is shaped to position the transducers 120 diametrically opposite, longitudinally displaced from each other, and facing each other squarely.

The inside surface of the housing 510, including bottom surface 518 and optionally side surfaces 522, may have a plurality of projections 520 such a longitudinal ridges as shown. The projections 520 may be distributed along the interior surface of the housing 510 with the exception of the channel 516 to avoid interference with illumination from the transducers 120. The projections 520 reduce the amount of contact area between the blood vessel and the interior surface of the housing 510, thereby reducing the likelihood of vessel erosion.

With reference to FIGS. 6A-6E, a sixth embodiment of a housing design 610 for the flow probe 100 is shown schematically. FIG. 6A provides a perspective view, FIG. 6B provides a top view, FIG. 6C provides a front view, FIG. 6D provides a side view, and FIG. 6E provides a detailed view taken along line E in FIG. 6C. With specific reference to FIG. 6B, the housing 610 includes two receptacles 612 to receive a coupling member, such as coupling member 730 shown in FIG. 7. Housing 610 also includes a strain relief 614 through which the cable 150 may extend.

A transducer channel 616 extends along the inside surface of the housing 610, including bottom surface 618 and side surfaces 622. Channel 616 functions as a window for the ultrasonic transducers 120 through which the transducers 120 illuminate the blood vessel therein. Channel 616 is shaped to position the transducers 120 diametrically opposite, longitudinally displaced from each other, and facing each other squarely.

The inside surface of the housing 610, including bottom surface 618 and optionally side surfaces 622, may have a plurality of recesses 620 such a plurality of longitudinal depressions as shown. The recesses 620 may be distributed along the interior surface of the housing 610, and may optionally extend through channel 616. The recesses 620 reduce the amount of contact area between the blood vessel and the interior surface of the housing 610, thereby reducing the likelihood of vessel erosion.

With reference to FIGS. 7A-7D, an alternative embodiment of a coupling member 730 for the flow probe 100 is shown schematically. FIG. 7A provides a perspective view, FIG. 7B provides a top view, FIG. 7C provides a side view and FIG. 7D provides a bottom view. With specific reference to FIG. 7B, the coupling member 730 may include a first end portion 732 and a second end portion 734 that releasably fit (e.g., snap-fit) into the corresponding receptacles on the housing to provide a secure connection therebetween. The first end portion 732 may include two pin or ball projections that pivotally fit into corresponding sockets on the housing, thereby defining a hinge mechanism allowing the coupling member to pivot relative to the housing. The second end portion 734 may include a slot 737 to allow the sides of the second end portion 734 to be manually compressed together to facilitate insertion into the corresponding receptacle in the housing, and subsequently released to secure the end portion 734 therein.

The coupling member 730 may also include indentations 738 to facilitate grasping and manipulation of the coupling member 730 with a tool such as a forceps. Alternatively, the coupling member 730 may incorporate other mating geometers to facilitate grasping and manipulation such as those described in Doten et al. '430.

When connected to the housing, the coupling member 730 partially surrounds the blood vessel and thus incorporates a curved portion 740 to conform thereto. The cured portion 740 may optionally have any of the features (tapers, projections, recesses, etc.) discussed with reference to the interior surface of the housing designs described previously.

With reference to FIG. 8, another embodiment of a flow probe 800 is shown schematically. Flow probe 800 is substantially similar to flow probe 100 described previously, except that the coupling member 830 is adjustable to atraumatically accommodate blood vessels of different size. The coupling member 830 may be made adjustable, for example, by providing a fixed portion 832 and an adjustable portion 834. Fixed portion 832 may be substantially the same as coupling member 730 described previously, except that cured portion 740 may be incorporated into the adjustable portion 834. A rod 836 may be connected to the adjustable portion 834 and slidably disposed a lock collar 838 connected to the fixed portion 832 the lock collar 838 may include a set screw or pin that engages the rod 836 therein to lock the adjustable portion 834 relative to the fixed portion 832 once the desired size of the probe opening has been determined. By correctly sizing the probe 800 for the target vessel, the probe may provide a better fit that is less susceptible to movement relative to the blood vessel and therefore less susceptible to vessel erosion.

With reference to FIG. 9, yet another embodiment of a flow probe 900 is shown schematically. Flow probe 900 is substantially similar to flow probe 100 described previously, except that a tissue in-growth promoting material 940 is wrapped about the housing portion 910 and coupling member 930. Material 940 may comprise, for example, a knitted, woven or non-woven fabric and/or a mesh. The fabric and/or mesh may comprise a polymeric material (e.g., ePTFE, PTFE, polyester, and the like). The material 940 may be selectively wrapped about the bottom portion of the housing and the top portion of the coupling member to avoid interference with the illumination of the blood vessel by the transducers 120 (not shown). The material 940 promotes tissue in-growth to more stably secure the probe 900 to the blood vessel, thereby reducing the likelihood of vessel erosion.

From the foregoing, it will be apparent to those skilled in the art that the present invention provides, in exemplar no-limiting embodiments, flow probe designs that are relatively atraumatic to blood vessels and therefore are suitable for chronic implantation. Further, those skilled in the art will recognize that the present invention may be manifested in a variety of forms other than the specific embodiments described and contemplated herein. Accordingly, departs in form and detail may be made without departing from the scope and spirit of the present invention as described in the appended claims. The entire disclosure of all patents and patent applications mentioned in this document are hereby incorporated by reference in their entirety.

What is claimed is:

1. A flow probe for measurement of fluid flow in a conduit, comprising: a housing configured to at least partially surround the conduit, the housing having an open portion configured to receive the conduit therethrough; at least one flow sensor disposed in the housing; and a coupling member connected to the housing, the coupling member configured to at least partially surround the conduit and to close the open portion of the housing.

2. A device as in claim 1, wherein the fluid comprises blood.

3. A device as in claim 1, wherein the conduit comprises a blood vessel.

4. A device as in claim 1, wherein the at least one flow sensor comprises one or more ultrasound transducers.

5. A device as in claim 4, wherein the one or more ultrasound transducers are disposed in the housing diametrcally opposite and longitudinally spaced from each other.

6. A device as in claim 5, wherein the housing defines at least one window for the ultrasound transducers.

7. A device as in claim 6, wherein the at least one window comprises an interior groove extending at least partially around the conduit.

8. A device as in claim 7, wherein the housing comprises a first material and a second material is disposed in the interior groove.

9. A device as in claim 8, wherein the second material substantially fills the interior groove.

10. A device as in claim 8, wherein the second material has an ultrasonic property that is similar to an ultrasonic property of blood vessel tissue.

11. A device as in claim 8, wherein the first material has a first ultrasonic property and the second material has second ultrasonic property that is different from the first ultrasonic property.

12. A device as in claim 8, wherein the second material has an acoustic impedance that is similar to an acoustic impedance of blood vessel tissue.

13. A device as in claim 8, wherein the first material has a first acoustic impedance and the second material has second acoustic impedance that is different from the first acoustic impedance.

14. A device as in claim 8, wherein the second material has a propagation constant that is similar to a propagation constant of blood vessel tissue.

15. A device as in claim 8, wherein the first material has a first propagation constant and the second material has second propagation constant that is different from the first propagation constant.

16. A device as in claim 8, wherein the second material has an attenuation and propagation constant that is similar to an attenuation and propagation constant of blood vessel tissue.

17. A device as in claim 8, wherein the first material has a first attenuation and propagation constant and the second material has second attenuation and propagation constant that is different from the first attenuation and propagation constant.

18. A device as in claim 1, wherein the housing and coupling member comprise biocompatible materials.

19. A device as in claim 1, wherein the coupling member includes two ends that are releasably connectable to the housing, and wherein at least one of the ends is pivotable when connected to the housing.

20. A device as in claim 1, wherein the housing has an interior surface that interfaces with the conduit, the interior surface having a concave geometry in lateral cross section and a convex geometry in longitudinal cross section.

21. A device as in claim 1, wherein the housing has an interior surface that interfaces with the conduit, the interior surface having a concave curvature in lateral cross section and a convex taper in longitudinal cross section.

22. A device as in claim 1, wherein the housing has an interior surface that interfaces with the conduit, the interior surface having a substantially square geometry in lateral cross section.

23. A device as in claim 1, wherein the housing has an interior surface that interfaces with the conduit, the interior surface having a plurality of projections.

24. A device as in claim 1, wherein the housing has an interior surface that interfaces with the conduit, the interior surface having a plurality of recesses.

25. A device as in claim 1, further comprising a layer of material surrounding at least a portion of the housing and the coupling member, wherein the layer of material promotes tissue in-growth.

26. A device as in claim 1, further comprising a layer of material overlaying a portion of the housing, wherein the layer of material promotes tissue in-growth.

27. A device as in claim 26, wherein the layer of material comprises a polymeric fabric.

28. A device as in claim 26, wherein the layer of material comprises a polymeric mesh.

29. A device as in claim 1, further comprising a layer of material overlaying a portion of the coupling member, wherein the layer of material promotes tissue in-growth.

30. A device as in claim 1, wherein the housing and the coupling define an interior size, and wherein a portion of the coupling member is adjustable to change the size.

31. A system for measurement of fluid flow in a conduit, comprising: an implantable flow probe as defined in claim 1; an implantable flow meter circuit connected to the flow probe; an implantable telemetry circuit connected to the flow meter; and a remote telemetry device in wireless communication with the implantable telemetry unit.

32. A method of measuring blood flow in a blood vessel, comprising: providing a flow probe as defined in claim 1; providing a flow meter circuit connected to the flow probe; providing a telemetry circuit connected to the flow meter; and implanting the flow probe about the blood vessel.
